(19) United States
(12) Patent Application Publication
(10) Pub. No.: US 2026/0364137 A1
(43) Pub. Date: Nov. 19, 2026

(54) MEN'S HEALTH SUPPLEMENT COMPOSITIONS AND USES THEREOF

(71) Applicant: Melaleuca, Inc., Idaho Falls, ID (US)

(72) Inventors: Jordan Joy, Idaho Falls, ID (US); Nasser A, Fredj, Idaho Falls, ID (US)

(21) Appl. No.: 19/675,714

(22) Filed: May 13, 2026

Related U.S. Application Data
(60) Provisional application No. 63/804,922, filed on May 13, 2025.

(51) Int. Cl. A61K 36/87; A61K 9/20; A61K 9/48; A61K 31/7048; A61K 36/29; A61K 36/31; A61K 36/48; A61K 36/81; A61P 15/00

(52) U.S. Cl. CPC A61K 36/87; A61K 9/2009; A61K 9/2013; A61K 9/2054; A61K 9/485; A61K 9/4858; A61K 9/4866; A61K 31/7048; A61K 36/29; A61K 36/31; A61K 36/48; A61K 36/81; A61P 15/00

(57) ABSTRACT

This document provides men's health supplement compositions and uses thereof. For example, men's health supplement compositions provided herein can include a grape seed extract, a Eurycoma (e.g., E. longifolia) extract, a Lepidium (e.g., L. meyenii) extract, an Epimedium (e.g., E. sagittatum) extract, and a fenugreek extract, an ashwagandha extract, or a fenugreek extract and an ashwagandha extract.

### CROSS-REFERENCE TO RELATED APPLICATIONS

This application claims priority from U.S. Provisional Application Ser. No. 63/804,922, filed May 13, 2025. The disclosure of the prior application is considered part of (and is incorporated by reference in) the disclosure of this application.

### TECHNICAL FIELD

This document relates to men's health supplement compositions and uses thereof. For example, this document relates to men's health supplement compositions that include a grape seed extract, a Eurycoma longifolia extract, a Lepidium meyenii extract, an Epimedium sagittatum extract, and a fenugreek extract, an ashwagandha extract, or a fenugreek extract and an ashwagandha extract.

### BACKGROUND

Beginning as early as age 30, men's testosterone levels naturally begin to decline by about 1% to about 2% per year, which can negatively affect men's overall quality of life. Testosterone is a hormone that can promote muscle growth and strength, regulate fat storage, stimulate growth of facial and body hair, and support strong bones, red blood cell production, mood regulation, sperm production, and libido. In general, men's testosterone levels are lower now than they were a generation ago due to a variety of factors, including lack of exercise, unhealthy eating and sleeping habits, and exposure to higher levels of endocrine-disrupting chemicals such as phthalates, pesticides, plastics, and per- and polyfluoroalkyl substances (PFAS). Thus, products that support testosterone levels are highly desirable.

### SUMMARY

This document is based, at least in part, on the discovery that compositions containing grape seed extract, Eurycoma longifolia extract, Lepidium meyenii extract, Epimedium sagittatum extract, and fenugreek extract and/or ashwagandha extract can have beneficial effects on men's health. Provided herein are supplement compositions that include a grape seed extract, a Eurycoma extract, a Lepidium extract, an Epimedium extract, and a fenugreek extract, an ashwagandha extract, or a fenugreek extract and an ashwagandha extract. Also provided herein are methods for using the compositions described herein for improving men's health. In some embodiments, the supplement compositions can further include ingredients, such as one or more excipients (e.g., microcrystalline cellulose, silica, and/or magnesium stearate).

In a first aspect, this document features a supplement composition for improving men's health. The supplement composition can include, consist of, or consist essentially of a grape seed extract, a Eurycoma longifolia extract, a Lepidium meyenii extract, an Epimedium sagittatum extract, and a fenugreek extract, an ashwagandha extract, or a fenugreek extract and an ashwagandha extract. In some embodiments, the fenugreek seed extract is present in the composition in an amount of from about 50 mg to about 5000 mg per serving of the supplement composition. In some embodiments, the Eurycoma longifolia extract is present in the composition in an amount of from about 5 mg to about 500 mg per serving of the supplement composition. In some embodiments, the Epimedium sagittatum extract is present in the composition in an amount of from about 10 mg to about 600 mg per serving of the supplement composition. In some embodiments, the Lepidium meyenii extract is present in the composition in an amount of from about 10 mg to about 1000 mg per serving of the supplement composition. In some embodiments, the grape seed extract is present in the composition in an amount of from about 10 mg to about 2000 mg per serving of the supplement composition. In some embodiments, the ashwagandha extract is present in the composition in an amount of from about 250 mg to about 2000 mg per serving of the supplement composition. In some embodiments, the microcrystalline cellulose is present in the composition in an amount of from about 1 mg to about 200 mg per serving of the supplement composition. In some embodiments, the silica is present in the composition in an amount of from about 0.1 mg to about 25 mg per serving of the supplement composition. In some embodiments, the magnesium stearate is present in the composition in an amount of from about 1 mg to about 100 mg per serving of the supplement composition. In some embodiments, the ashwagandha extract is an ashwagandha root extract.

In some embodiments, the supplement composition is formulated as one or more tablets, one or more capsules, and/or one or more soft gels. In some embodiments, one or more tablets, one or more capsules, and/or one or more soft gels can comprise a full serving of the composition.

In another aspect, this document provides methods of increasing testosterone levels in a male mammal. This document also features methods of improving health in a male mammal. Also, provided herein are methods of improving vitality in a male mammal. In some embodiments, the methods can include administering the supplement compositions described herein to a mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description, and from the claims.

### DETAILED DESCRIPTION

This document provides supplement compositions that contain (a) a grape seed extract, (b) a Eurycoma (e.g., Eurycoma longifolia) extract, (c) a Lepidium (e.g., Lepidium meyenii) extract, (d) an Epimedium (e.g., Epimedium sagittatum) extract, and (e) a fenugreek extract, an ashwagandha extract, or a fenugreek extract and an ashwagandha extract. In some embodiments, a supplement composition provided herein can contain a grape seed extract, a Eurycoma extract, a Lepidium extract, an Epimedium extract, a fenugreek extract, and an ashwagandha extract. In some embodiments, a supplement composition provided herein can contain a grape seed extract, a Eurycoma extract, a Lepidium extract, an Epimedium extract, and a fenugreek extract. In some embodiments, a supplement composition can contain a grape seed extract, a Eurycoma extract, a Lepidium extract, an Epimedium extract, and an ashwagandha extract. In some embodiments, a supplement composition provided herein can further contain one or more additional ingredients, such as one or more excipients (e.g., microcrystalline cellulose, silica, and/or magnesium stearate). For example, a supplement composition provided herein can contain a grape seed extract, a Eurycoma extract, a Lepidium extract, an Epimedium extract, and a fenugreek extract, an ashwagandha extract, or a fenugreek extract and an ashwagandha extract, as well as one or more of microcrystalline cellulose, silica, and magnesium stearate. In some embodiments, a supplement composition provided herein can contain a grape seed extract, a Eurycoma longifolia extract, a Lepidium meyenii extract, an Epimedium sagittatum extract, and a fenugreek extract, an ashwagandha extract, or a fenugreek extract and an ashwagandha extract, as well as one or more excipients (e.g., microcrystalline cellulose, silica, and/or magnesium stearate).

### Grape Extract

The supplement compositions provided herein can contain grape seed extract, grape skin extract, or a combination of both grape seed extract and grape skin extract. For example, the compositions provided herein can contain a blend of grape seed and grape skin extracts. In some cases, the supplement compositions can contain a pre-mixed combination of grape seed extract and/or grape skin extract with additional ingredients (e.g., as in PROVEXCV®; Melaleuca, Inc., Idaho Falls, ID). Without being bound by a particular mechanism, grape skin and/or grape skin extract can promote healthy blood flow by supporting relaxation and widening of blood vessels.

The supplement compositions provided herein can contain any appropriate amount of grape seed extract. In some embodiments, grape seed extract and/or grape skin extract can be present in a supplement composition provided herein in an amount of about 10% to about 35% (e.g., about 10% to about 14%, about 14% to about 18%, about 18% to about 21%, about 21% to about 25%, about 25% to about 28%, about 28% to about 32%, or about 32% to about 35%) w/w of a supplement composition provided herein. For example, grape seed extract can be present in an amount of about 10% to about 35% (e.g., about 10% to about 14%, about 14% to about 18%, about 18% to about 21%, about 21% to about 25%, about 25% to about 28%, about 28% to about 32%, or about 32% to about 35%) w/w of a supplement composition provided herein.

In some embodiments, the supplement compositions described herein can include grape seed and/or grape skin extract in an amount of about 10 mg to about 2000 mg per serving of a supplement composition. For example, grape seed extract can be present in an amount of about 10 mg to about 210 mg, about 210 mg to about 410 mg, about 410 mg to about 605 mg, about 605 mg to about 805 mg, about 805 mg to about 1005 mg, about 1005 mg to about 1205 mg, about 1205 mg to about 1405 mg, about 1405 mg to about 1600 mg, about 1600 mg to about 1800 mg, or about 1800 mg to about 2000 mg per serving of a supplement composition provided herein. In some embodiments, grape seed extract can be present in an amount of at least about 10 mg per serving of a supplement composition provided herein (e.g., at least about 20 mg, at least about 30 mg, at least about 40 mg, at least about 50 mg, at least about 60 mg, at least about 70 mg, at least about 80 mg, at least about 90 mg, at least about 100 mg, at least about 200 mg, at least about 300 mg, at least about 400 mg, at least about 500 mg, at least about 600 mg, at least about 700, at least about 800 mg, at least about 900 mg, or at least about 1000 mg per serving of a supplement composition provided herein).

In some embodiments, the supplement compositions provided herein can include, without limitation, one or more fat-soluble antioxidants, phenolic compounds, and/or water soluble antioxidants (e.g., instead of grape seed and/or grape skin extract). For example, a supplement composition can include at least one fat-soluble antioxidant, at least one phenolic compound, and at least one water soluble antioxidant. Examples of fat-soluble antioxidants that can be included within a supplement composition include, without limitation, tocopherols such as α-tocopherol, β-tocopherol, γ-tocopherol, and δ-tocopherol, carotenoids such as α-carotene, β-carotene, cryptoxanthin, zeaxanthin, lutein, and lycopene. In some cases, the supplement compositions can include a carotenoid blend that includes α-carotene, β-carotene, cryptoxanthin, zeaxanthin, lutein, and lycopene. Examples of phenolic compounds that can be included within a supplement composition include, without limitation, tyrosol, hydroxytyrosol, verbascoside, olive extract containing tyrosol, hydroxytyrosol, oleuropein, verbascoside and/or, oligomeric proanthocyanidins, grape seed extract containing oligomeric proanthocyanidins, catechin, epicatechin, gallic acid. Examples of water soluble antioxidant that can be included within a supplement composition includes, without limitation, Vitamin C (ascorbic acid). In some cases, a supplement composition can be formulated into a single composition (e.g., a capsule) having one or more of α-tocopherol, β-tocopherol, γ-tocopherol, δ-tocopherol, α-carotene, β-carotene, cryptoxanthin, zeaxanthin, lutein, lycopene, olive fruit extract, grape seed extract, and vitamin C.

In some embodiments, the supplement compositions provided herein can include, without limitation, one or more vasodilators, one or more flavanols, or one or more amino acids. Examples of vasodilators that can be included in the supplement compositions include, without limitation, beets, arugula, spinach, or other nitrate-containing plants. zeaxanthin, lutein, lycopene, olive fruit extract, grape seed extract, and vitamin C. Examples of flavanols that can be included in the supplement compositions include, without limitation, chocolate or citrus fruits. Examples of amino acids that can be included in the supplement compositions include, without limitation, arginine or citrulline. In some cases, the supplement compositions provided herein can include gingko, turmeric, garlic, or pine bark extract.

In some cases, the ingredients can be obtained from sources that were not subjected to a fermentation process. For example, unfermented grape seed extract and/or unfermented grape skin extract can be used as ingredients for a supplement composition. Such unfermented ingredients can be obtained from any supplier such as Polyphenolics (Canandaigua, NY). Briefly, fermentation processes are used during the production of wine. If an ingredient is obtained from a supplier involved in wine production, then the ingredient could be a fermented ingredient. For example, a fermented ingredient, such as fermented grape seed extract or fermented grape skin extract, can be any ingredient isolated from a source, such as grapes, that was subjected to fermentation. In contrast, an unfermented ingredient can be any ingredient isolated from a source not subjected to fermentation.

### Eurycoma

The supplement compositions provided herein include a Eurycoma (e.g., E. longifolia) extract. E. longifolia, commonly known as tongkat ali, is a flowering plant of the family Simaroubaceae, native to Indonesia, Malaysia, Vietnam, Cambodia, Myanmar, Laos, and Thailand. Eurycoma (e.g., E. longifolia) extracts can be prepared from the leaves, roots, stem, bark, or any other aerial part of the Eurycoma (e.g., E. longifolia) plant, and can be in the form of a liquid (e.g., an aqueous solution), a solid (e.g., a particulate or a powder), or a semi-solid form (e.g., a gel or an emulsion). Any appropriate method can be used to obtain a Eurycoma (e.g., E. longifolia) extract for use in the supplement compositions provided herein. For example, E. longifolia root powder can be prepared by drying and grinding the root of the E. longifolia plant. In some cases, an E. longifolia extract can be obtained at an extraction ratio of about 100:1.

The supplement compositions provided herein can contain any appropriate amount of Eurycoma (e.g., E. longifolia) extract (e.g., E. longifolia root extract, leaf extract, and/bark extract). In some embodiments, Eurycoma (e.g., E. longifolia) extract can be present in an amount of about 0.5% to about 50% w/w of a supplement composition provided herein. For example, E. longifolia extract can be present in an amount of about 0.5% to about 5%, about 5% to about 10%, about 10% to about 15%, about 15% to about 20%, about 20% to about 25%, about 25% to about 30%, about 30% to about 35%, about 35% to about 40%, about 40% to about 45%, or about 45% to about 50% w/w of a supplement composition provided herein. In some embodiments, E. longifolia extract can be present in an amount of about 0.5% to about 10% w/w of a supplement composition provided herein. In some embodiments, E. longifolia extract is present can be an amount of about 0.5% to about 2%, about 2% to about 5%, about 5% to about 8%, or about 8% to about 10% w/w of a supplement composition provided herein.

In some embodiments, Eurycoma (e.g., E. longifolia) extract can be present in a supplement composition provided herein in an amount of about 5 mg to about 500 mg per serving of a supplement composition provided herein. For example, Eurycoma (e.g., E. longifolia) extract can be present in an amount of about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, about 45 mg to about 50 mg, or about 50 mg to about 55 mg per serving of a supplement composition provided herein. In some embodiments, E. longifolia extract can be present in an amount of about 20 mg to about 80 mg per serving of a supplement composition provided herein. For example, E. longifolia extract can be present in an amount of about 20 mg to about 25 mg, about 25 mg to about 30 mg, about 30 mg to about 35 mg, about 35 mg to about 40 mg, about 40 mg to about 45 mg, about 45 mg to about 50 mg, about 50 mg to about 55 mg, about 55 mg to about 60 mg, about 60 mg to about 65 mg, about 65 mg to about 70 mg, about 70 mg to about 75 mg, or about 75 mg to about 80 mg per serving of a supplement composition provided herein.

### Lepidium

The supplement compositions provided herein include a Lepidium (e.g., L. meyenii) extract. L. meyenii extract can be prepared from, for example, L. meyenii root. L. meyenii, also known as Maca, is an edible root native to Andean region. Lepidium meyenii is rich in fatty acids, fiber, vitamin C, essential amino acids, minerals such as copper, iron, or calcium. A Lepidium (e.g., L. meyenii) extract can be in the form of a liquid (e.g., an aqueous solution), a solid (e.g., a particulate or a powder), or a semi-solid form (e.g., a gel or an emulsion). Any appropriate method can be used to obtain a Lepidium (e.g., L. meyenii) extract for use in the supplement compositions provided herein. For example, L. meyenii root powder can be prepared by boiling, baking, drying, and grinding the root of the L. meyenii plant. In some cases, L. meyenii extract can be obtained at an extraction ratio of about 20:1.

The supplement compositions provided herein can contain any appropriate amount of Lepidium (e.g., L. meyenii) extract. In some embodiments, Lepidium (e.g., L. meyenii) extract can be present in an amount of about 1% to about 25% w/w of a supplement composition provided herein. For example, L. meyenii extract can be present in an amount of about 1% to about 5%, about 5% to about 10%, about 10% to about 15%, about 15% to about 20%, or about 20% to about 25% w/w of a supplement composition provided herein. In some embodiments, L. meyenii extract can be present in an amount of about 5% to about 20% w/w of a supplement composition provided herein. In some embodiments, L. meyenii extract can be present in an amount of about 5% to about 6%, about 6% to about 7%, about 7% to about 8%, about 8% to about 9%, about 9% to about 10%, about 10% to about 11%, about 11% to about 12%, about 12% to about 13%, about 13% to about 14%, or about 14% to about 20% w/w of a supplement composition provided herein.

In some embodiments, Lepidium (e.g., L. meyenii) extract can be present in an amount of about 10 mg to about 1000 mg per serving of a supplement composition provided herein. For example, Lepidium (e.g., L. meyenii) extract can be present in an amount of about 10 mg to about 110 mg, about 110 mg to about 210 mg, about 210 mg to about 310 mg, about 310 mg to about 410 mg, about 410 mg to about 510 mg, about 510 mg to about 610 mg, about 610 mg to about 710 mg, about 710 mg to about 810 mg, about 810 mg to about 910 mg, or about 910 mg to about 1000 mg per serving of a supplement composition provided herein. In some embodiments, L. meyenii extract can be present in an amount of about 50 mg to about 200 mg per serving of a supplement composition provided herein. For example, L. meyenii extract can be present in an amount of about 50 mg to about 60 mg, about 60 mg to about 70 mg, about 70 mg to about 80 mg, about 80 mg to about 90 mg, about 90 mg to about 100 mg, about 100 mg to about 110 mg, about 110 mg to about 120 mg, about 120 mg to about 130 mg, about 130 mg to about 140 mg, about 140 mg to about 150 mg, or about 150 mg to about 200 mg per serving of a supplement composition provided herein.

### Epimedium

The supplement compositions provided herein also include Epimedium (e.g., E. sagittatum) extract. Epimedium sagittatum, also known as Horny Goat Weed, is a flowering plant in the Berberidaceae family, and is native to Japan and Korea. An Epimedium (e.g., E. sagittatum) extract can be a leaf extract, a whole plant extract, or a combination of both leaf extract and whole plant extract. In some embodiments, an E. sagittatum extract can be a whole plant extract. Any appropriate method can be used to obtain an Epimedium (e.g., E. sagittatum) extract for use in the supplement compositions provided herein. For example, E. sagittatum extract can be prepared by drying and grinding the leaves of the E. sagittatum plant. In some cases, a diluted ethanol solution can be used as an extraction solvent to obtain an E. sagittatum extract.

The supplement compositions provided herein can contain any appropriate amount of Epimedium (e.g., E. sagittatum) extract. For example, Epimedium (e.g., E. sagittatum) extract can be present in an amount of about 1% to about 25% w/w of a supplement composition provided herein. For example, E. sagittatum extract can be present in an amount of about 1% to about 5%, about 5% to about 10%, about 10% to about 15%, about 15% to about 20%, or about 20% to about 25% w/w of a supplement composition provided herein. In some embodiments, E. sagittatum extract can be present in an amount of about 1% to about 10% w/w of a supplement composition provided herein. In some embodiments, E. sagittatum extract can be present in an amount of about 1% to about 2%, about 2% to about 3%, about 3% to about 4%, about 4% to about 5%, about 4% to about 6%, about 5% to about 6%, about 5.5% to about 6%, about 6% to about 7%, about 7% to about 8%, about 8% to about 9%, or about 9% to about 10% w/w of a supplement composition provided herein.

In some embodiments, Epimedium (e.g., E. sagittatum) extract can be present in an amount of about 10 mg to about 600 mg per serving of a supplement composition provided herein. For example, Epimedium (e.g., E. sagittatum) extract can be present in an amount of about 10 mg to about 20 mg, about 20 mg to about 30 mg, about 30 mg to about 35 mg, about 35 mg to about 40 mg, about 40 mg to about 50 mg, about 45 mg to about 50 mg, about 50 mg to about 55 mg, about 50 mg to about 60 mg, about 55 mg to about 60 mg, about 60 mg to about 100 mg, about 100 mg to about 200 mg, about 200 mg to about 300 mg, about 300 mg to about 400 mg, about 400 mg to about 500 mg, or about 500 mg to about 600 mg per serving of a supplement composition provided herein. In some embodiments, E. sagittatum extract can be present in an amount of about 20 mg to about 80 mg per serving of a supplement composition provided herein. For example, E. sagittatum extract can be present in an amount of about 20 mg to about 30 mg, about 30 mg to about 40 mg, about 40 mg to about 50 mg, about 45 mg to about 50 mg, about 50 mg to about 60 mg, about 55 mg to about 60 mg, about 60 mg to about 70 mg, or about 70 mg to about 80 mg per serving of a supplement composition provided herein.

In some embodiments, the supplement compositions provided herein include Epimedium (e.g., E. sagittatum) whole plant extract that contains icariin. In some embodiments, an E. sagittatum whole plant extract can contain icariin in an amount of at least about 5%. For example, an E. sagittatum whole plant extract in a supplement composition provided herein can contain icariin in an amount of at least about 7%, at least about 10%, at least about 12%, at least about 15%, at least about 17%, at least about 20%, at least about 22%, at least about 25%, at least about 27%, or at least about 30%. In some embodiments, an E. sagittatum whole plant extract present in a supplement composition provided herein can contain about 20% icariin. In some embodiments, an E. sagittatum whole plant extract present in a supplement composition provided herein can contain icariin in an amount of about 2% to about 50%. For example, an E. sagittatum whole plant extract present in a supplement composition provided herein can contain icariin in an amount of about 2% to about 5%, about 5% to about 10%, about 10% to about 15%, about 15% to about 20%, about 20% to about 25%, about 25% to about 30%, about 30% to about 35%, about 35% to about 40%, about 40% to about 45%, or about 45% to about 50%. In some embodiments, an E. sagittatum whole plant extract present in a supplement composition provided herein can contain icariin in an amount of about 10% to about 30%. For example, an E. sagittatum whole plant extract present in a supplement composition provided herein can contain icariin in an amount of 10% to about 15%, about 15% to about 20%, about 20% to about 25%, or about 25% to about 30%.

The supplement compositions provided herein can include icariin sourced from any appropriate species of the Epimedium genus. For example, the supplement compositions provided herein can include an icariin sourced from E. sagittatum, E. grandiflorum, E. pubescens, E. koreanum, or E. brevicornum. In some embodiments, a whole plant extract of an Epimedium species (e.g., E. sagittatum, E. grandiflorum, E. pubescens, E. koreanum, or E. brevicornum) can contain icariin in an amount of at least about 5%. For example, an Epimedium whole plant extract in a supplement composition provided herein can contain icariin in an amount of at least about 7%, at least about 10%, at least about 12%, at least about 15%, at least about 17%, at least about 20%, at least about 22%, at least about 25%, at least about 27%, or at least about 30%. In some embodiments, the whole plant extract present in a supplement composition provided herein can contain about 20% icariin. In some embodiments, an Epimedium whole plant extract present in a supplement composition provided herein can contain icariin in an amount of about 2% to about 50%. For example, an Epimedium whole plant extract present in a supplement composition provided herein can contain icariin in an amount of about 2% to about 5%, about 5% to about 10%, about 10% to about 15%, about 15% to about 20%, about 20% to about 25%, about 25% to about 30%, about 30% to about 35%, about 35% to about 40%, about 40% to about 45%, or about 45% to about 50%. In some embodiments, an Epimedium whole plant extract present in a supplement composition provided herein can contain icariin in an amount of about 10% to about 30%. For example, an Epimedium whole plant extract present in a supplement composition provided herein can contain icariin in an amount of 10% to about 15%, about 15% to about 20%, about 20% to about 25%, or about 25% to about 30%.

### Fenugreek

In some cases, the supplement compositions provided herein can include fenugreek. For example, a fenugreek extract can be present in a supplement composition provided herein instead of, or in addition to, an ashwagandha extract. Fenugreek (Trigonella foenum-graecum) is an annual leguminous herb that has been traditionally used in India, China, Egypt, and some parts of Europe for medicinal and rejuvenating properties. Any appropriate method can be used to obtain a fenugreek extract for use in the supplement compositions provided herein. In some embodiments, fenugreek extract can be obtained from fenugreek seed. For example, fenugreek seed extract can be prepared by cleaning, sorting, and pulverizing the fenugreek seed.

The supplement compositions provided herein can contain any appropriate amount of fenugreek extract. In some embodiments, fenugreek extract can present in an amount of about 5% to about 90% w/w of a supplement composition provided herein. For example, fenugreek extract can be present in an amount of about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, or about 85% to about 90% w/w of a supplement composition provided herein. In some embodiments, fenugreek extract can be present in an amount of about 25% to about 75% w/w of a supplement composition provided herein. In some embodiments, fenugreek extract can be present in an amount of about 25% to about 35%, about 35% to about 45%, about 45% to about 50%, about 45% to about 55%, about 55% to about 65%, or about 65% to about 75% w/w of a supplement composition provided herein.

In some embodiments, a supplement composition provided herein can contain an amount of fenugreek seed extract that is equivalent to at least about 1500 fenugreek seeds (e.g., at least about 1500 fenugreek seeds, at least about 2000 fenugreek seeds, at least about 2500 fenugreek seeds, or at least about 3000 fenugreek seeds). For example, fenugreek extract can be present in an amount of about 50 mg to about 5000 mg per serving of a supplement composition provided herein. For example, fenugreek extract can be present in an amount of about 50 mg to about 550 mg, about 550 mg to about 1050 mg, about 1050 mg to about 1550 mg, about 1550 mg to about 2050 mg, about 2050 mg to about 2550 mg, about 2550 mg to about 3050 mg, about 3050 mg to about 3550 mg, about 3550 mg to about 4050 mg, about 4050 mg to about 4550 mg, or about 4550 mg to about 5000 mg per serving of a supplement composition provided herein. In some embodiments, fenugreek extract can be present in an amount of about 250 mg to about 750 mg per serving of a supplement composition provided herein. For example, fenugreek extract can be present in an amount of about 250 mg to about 350 mg, about 350 mg to about 450 mg, about 450 mg to about 550 mg, about 550 mg to about 650 mg, or about 650 mg to about 750 mg per serving of a supplement composition provided herein.

In some embodiments, the supplement compositions can include fenugreek extract that contains a furostanolic saponin. For example, the furostanolic saponin in a fenugreek extract can be a protodioscin. In some embodiments, the supplement compositions can include fenugreek extract that contains protodioscin. In some embodiments, a fenugreek extract present in a supplement composition provided herein can contain protodioscin in an amount of at least about 5%. For example, a fenugreek extract present in a supplement composition provided herein can contain protodioscin in an amount of at least about 7%, at least about 10%, at least about 12%, at least about 15%, at least about 17%, at least about 20%, at least about 22%, at least about 25%, at least about 27%, or at least about 30%. In some embodiments, a fenugreek extract present in a supplement composition provided herein can contain about 20% protodioscin. In some embodiments, a fenugreek extract present in a supplement composition provided herein can contain protodioscin in an amount of about 2% to about 50%. For example, fenugreek extract can contain protodioscin in an amount of about 2% to about 5%, about 5% to about 10%, about 10% to about 15%, about 15% to about 20%, about 20% to about 25%, about 25% to about 30%, about 30% to about 35%, about 35% to about 40%, about 40% to about 45%, or about 45% to about 50%. In some embodiments, a fenugreek extract present in a supplement composition provided herein can contain protodioscin in an amount of about 10% to about 30%. For example, a fenugreek extract present in a supplement composition provided herein can contain protodioscin in an amount of 10% to about 15%, about 15% to about 20%, about 20% to about 25%, or about 25% to about 30%.

### Ashwagandha

In some cases, the supplement compositions provided herein can include ashwagandha. For example, an ashwagandha extract can be present in a supplement composition provided herein instead of, or in addition to, fenugreek. Ashwagandha (Withania somnifera) is an evergreen shrub found in tropical and subtropical areas of Asia, Africa, and Europe, and has been traditionally used for reducing stress. In some embodiments, the supplement compositions provided herein can include ashwagandha whole root powder and/or ashwagandha extract.

In some embodiments, a supplement composition provided herein can include an ashwagandha whole root powder. A supplement composition provided herein can contain any appropriate amount of ashwagandha whole root powder. For example, ashwagandha whole root powder can be present in an amount of about 250 mg to about 2000 mg per serving of a supplement composition provided herein. For example, ashwagandha whole root powder can be present in an amount of about 250 mg to about 300 mg, about 300 mg to about 325 mg, about 325 mg to about 350 mg, about 350 mg to about 400 mg, about 400 mg to about 450 mg, about 450 mg to about 500 mg, about 500 mg to about 550 mg, about 525 mg to about 550 mg, about 550 mg to about 600 mg, about 575 mg to about 600 mg, about 600 mg to about 625 mg, about 600 mg to about 650 mg, about 625 mg to about 650 mg, about 650 mg to about 700 mg, about 675 mg to about 700 mg, about 700 mg to about 800 mg, about 800 mg to about 900 mg, about 900 mg to about 1100 mg, about 1100 mg to about 1300 mg, about 1300 mg to about 1500 mg, about 1500 mg to about 1750 mg, about 1750 mg to about 2000 mg per serving of a supplement composition provided herein. In some embodiments, a supplement composition provided herein can include an ashwagandha extract. An ashwagandha extract can be in the form of a liquid (e.g., an aqueous solution), a solid (e.g., a particulate or a powder), or a semi-solid form (e.g., a gel or an emulsion). Any appropriate method can be used to obtain an ashwagandha extract for use in the supplement compositions provided herein. For example, ashwagandha whole root powder can be prepared by drying and grinding the root of the ashwagandha plant. As another example, an alcohol (e.g., ethanol) solution can be used to obtain an ashwagandha extract from ashwagandha roots and/or ashwagandha leaves. In some embodiments, an ashwagandha extract present in a supplement composition provided herein can be an extract from ashwagandha leaves and/or ashwagandha roots. For example, an ashwagandha extract present in a supplement composition provided herein can be an ashwagandha root extract. An ashwagandha root extract can be an extract produced without using any ashwagandha leaves. In some embodiments, the proportions of compounds in an ashwagandha extract can be similar to their proportions found naturally (e.g., in ashwagandha leaves and/or ashwagandha roots). In some embodiments, a supplement composition provided herein can include ashwagandha whole root powder.

The supplement compositions provided herein can contain any appropriate amount of an ashwagandha extract. In some embodiments, an ashwagandha extract can be present in an amount of about 0.4% to about 50% w/w of a supplement composition provided herein. For example, an ashwagandha extract can be present in an amount of about 0.4% to about 5%, about 5% to about 10%, about 10% to about 15%, about 15% to about 20%, about 20% to about 25%, about 25% to about 30%, about 30% to about 35%, about 35% to about 40%, about 40% to about 45%, or about 45% to about 50% w/w of a supplement composition provided herein. In some embodiments, an ashwagandha extract can be present in an amount of about 8% to about 40% w/w of a supplement composition provided herein. In some embodiments, an ashwagandha extract can be present in an amount of about 5%, about 10%, about 15%, about 20%, about 25%, about 30%, about 35%, or about 40% w/w of a supplement composition provided herein.

In some embodiments, an ashwagandha extract can be present in an amount of about 5 mg to about 600 mg per serving of a supplement composition provided herein. For example, an ashwagandha extract can be present in an amount of about 5 mg to about 55 mg, about 55 mg to about 105 mg, about 105 mg to about 155 mg, about 155 mg to about 205 mg, about 205 mg to about 255 mg, about 255 mg to about 305 mg, about 305 mg to about 355 mg, about 355 mg to about 405 mg, about 405 mg to about 455 mg, about 455 mg to about 505 mg, about 505 mg to about 555 mg, or about 555 mg to about 600 mg per serving of a supplement composition provided herein.

Ashwagandha whole root powders and ashwagandha extracts can contain one or more withanolides. Withanolides include compounds consisting of a steroid backbone bound to a lactone or a lactone derivative. Non-limiting examples of a withanolide include withaferin A, salpichrolid A, nicandrenone-1, ixocarpalacton A, withaperuvin B, withangulatin C, acnistin I, physalin X, jaborosalactol 18, physalin C, jaborosalactone 7, salpichrolide G, jaborosalactone 37, jaborosalactone 43, subtrifloralactone E, tubonolide A, withametelinone, withaphysalin N, isocarpalactone B, perulactone C, jaborosalactone 15, subtrifloralactone K, taccalonolide A, jaborosalactol 24, physanolide A, exodeconolide A, withanoside VII, 27-hydroxywithanone, dihydrowithaferin A, viscosalactone B, withanoside IV, withanoside V, withaferin A, 12-deoxywithastramonolide, withanolide A, withanone, and withanolide B. In some embodiments, the one or more withanolides include withaferin A.

In some embodiments, an ashwagandha whole root powder present in a supplement composition provided herein can contain one or more withanolides present in an amount of at least about 0.4%. For example, an ashwagandha whole root powder present in a supplement composition provided herein can contain at least about 0.5%, at least about 0.6%, at least about 0.7%, at least about 0.8%, at least about 0.9%, at least about 1%, or at least about 1.2% w/w of the ashwagandha whole root powder. In some embodiments, an ashwagandha whole root powder present in a supplement composition provided herein can contain one or more withanolides in an amount of about 0.5% to about 1.5% w/w of the ashwagandha whole root powder. For example, an ashwagandha whole root powder present in a supplement composition provided herein can contain one or more withanolides present in an amount of about 0.5% to about 0.8%, about 0.5% to about 1%, about 0.5% to about 1.2%, about 0.8% to about 1%, about 0.8% to about 1.2%, about 0.8% to about 1.5%, about 1% to about 1.2%, about 1% to about 1.5%, or about 1.2% to about 1.5% w/w of the ashwagandha whole root powder.

In some embodiments, an ashwagandha extract present in a supplement composition provided herein can contain one or more withanolides present in an amount of at least about 4%, e.g., at least about 5%, at least about 6%, at least about 7%, at least about 8%, at least about 9%, or at least about 10% w/w of the ashwagandha extract. In some embodiments, an ashwagandha extract present in a supplement composition provided herein can contain one or more withanolides present in an amount of about 1% to about 12% w/w of the ashwagandha extract. For example, an ashwagandha extract present in a supplement composition provided herein can contain one or more withanolides present in an amount of about 1% to about 2%, about 1% to about 3%, about 1% to about 4%, about 1% to about 5%, about 1% to about 6%, about 1% to about 7%, about 1% to about 8%, about 1% to about 9%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, about 9% to about 10%, or about 10% to about 12% w/w of the ashwagandha extract. In some embodiments, an ashwagandha extract present in a supplement composition provided herein can contain one or more withanolides present in an amount of about 5% w/w of the ashwagandha extract.

In some embodiments, one or more withanolides can be present in an amount of about 0.25 mg to about 30 mg per serving of a supplement composition provided herein. For example, one or more withanolides can be present in an amount of about 0.25 mg to about 1 mg, about 0.25 mg to about 5 mg, about 0.25 mg to about 10 mg, about 0.25 mg to about 15 mg, about 0.25 mg to about 20 mg, about 0.25 mg to about 25 mg, about 1 mg to about 5 mg, about 1 mg to about 10 mg, about 1 mg to about 15 mg, about 1 mg to about 20 mg, about 1 mg to about 25 mg, about 1 mg to about 30 mg, about 5 mg to about 10 mg, about 5 mg to about 15 mg, about 5 mg to about 20 mg, about 5 mg to about 25 mg, about 5 mg to about 30 mg, about 10 mg to about 15 mg, about 10 mg to about 20 mg, about 10 mg to about 25 mg, about 10 mg to about 30 mg, about 15 mg to about 20 mg, about 15 mg to about 25 mg, about 15 mg to about 30 mg, about 20 mg to about 25 mg, about 20 mg to about 30 mg, or about 25 mg to about 30 mg per serving of a supplement composition provided herein. In some embodiments, one or more withanolides can be present in an amount of about 5 mg to about 25 mg per serving of a supplement composition provided herein. In some embodiments, one or more withanolides can be present in an amount of about 5 mg, about 10 mg, about 15 mg, about 20 mg, or about 25 mg per serving of a supplement composition provided herein. In some embodiments, one or more withanolides can be present in an amount of about 6.25 mg per serving of a supplement composition provided herein.

In some embodiments, one or more withanolides can be present in an amount of about 0.04% to about 2% w/w of a supplement composition provided herein. For example, one or more withanolides can be present in an amount of about 0.04% to about 0.1%, about 0.1% to about 0.2%, about 0.2% to about 0.3%, about 0.3% to about 0.4%, about 0.4% to about 0.5%, about 0.5% to about 0.6%, about 0.6% to about 0.7%, about 0.7% to about 0.8%, about 0.8% to about 0.9%, about 0.9% to about 1%, about 1% to about 1.1%, about 1.1% to about 1.2%, about 1.2% to about 1.3%, about 1.3% to about 1.4%, about 1.4% to about 1.5%, about 1.5% to about 1.6%, about 1.6% to about 1.7%, about 1.7% to about 1.8%, about 1.8% to about 1.9%, or about 1.9% to about 2% w/w of a supplement composition provided herein. In some embodiments, one or more withanolides can be present in an amount of about 0.1%, about 0.2%, about 0.3%, about 0.4%, about 0.5%, about 0.6%, about 0.7%, about 0.8%, or about 1% w/w of a supplement composition provided herein. In some embodiments, one or more withanolides can be present in an amount of about 0.5% w/w of a supplement composition provided herein.

### Excipients

In some cases, a supplement composition provided herein can include one or more suitable excipients. For example, a supplement composition provided herein for oral administration (e.g., in the form of a powder, granules, one or more tablets, one or more capsules, syrups, one or more lozenges, or an aqueous or non-aqueous solution) can be prepared with any appropriate pharmaceutically acceptable excipients. Examples of suitable pharmaceutically acceptable excipients include, without limitation, those discussed below.

In some cases, a supplement composition provided herein can include one or more fillers (e.g., a filler having a water activity (Aw) of less than 0.2). Examples of suitable fillers include, without limitation, microcrystalline cellulose (MCC) 112, rice flour, maltodextrin (e.g., maltodextrin derived from any appropriate source such as rice, wheat, potato, corn, sorghum, cassava root, or any other starch-containing plant), mannitol, MCC 302, MCC 200 LM, MCC 101, starch, xylitol, sorbitol, hydroxyl propyl cellulose, gelatin, and dibasic calcium phosphate. In some embodiments, a supplement composition provided herein can include a silicified microcrystalline cellulose.

Microcrystalline cellulose (e.g., silicified microcrystalline cellulose) can be present in a supplement composition provided herein in any appropriate amount. In some embodiments, a silicified microcrystalline cellulose can be present in an amount of about 0.2% to about 30% w/w of a supplement composition provided herein. For example, a silicified microcrystalline cellulose can be present in an amount of about 0.2% to about 5%, about 5% to about 10%, about 10% to about 15%, about 15% to about 20%, about 20% to about 25%, or about 25% to about 30% w/w of a supplement composition provided herein. In some embodiments, a silicified microcrystalline cellulose can be present in an amount of about 0.2% to about 10% w/w of a supplement composition provided herein. In some embodiments, a silicified microcrystalline cellulose can be present in an amount of about 0.2% to about 0.5%, about 0.5% to about 1%, about 1% to about 2%, about 1% to about 1.5%, about 1.5% to about 2.5%, about 1.5% to about 2%, about 2% to about 3%, about 2% to about 2.5%, about 2.5% to about 3.5%, about 2.5% to about 3%, about 3% to about 4%, about 3% to about 3.5%, about 3.5% to about 4%, about 4% to about 6%, about 6% to about 8%, or about 8% to about 10% w/w of a supplement composition provided herein.

In some cases, a supplement composition provided herein can include about 0.5 mg to about 200 mg of silicified microcrystalline cellulose per serving of the supplement composition. For example, a supplement composition provided herein can contain about 0.5 mg to about 1 mg, about 0.5 mg to about 0.75 mg, about 0.75 mg to about 1 mg, about 1 mg, to about 1.5 mg, about 1 mg to about 1.25 mg, about 1.25 mg to about 1.5 mg, about 1.5 mg to about 2 mg, about 1.75 mg to about 2 mg, about 2 mg to about 2.5 mg, about 2.5 mg to about 3 mg, about 3 mg to about 5 mg, about 5 mg to about 30 mg, about 30 mg to about 55 mg, about 55 mg to about 80 mg, about 80 mg to about 105 mg, about 105 mg to about 135 mg, about 135 mg to about 160 mg, about 160 mg to about 185 mg, or about 185 mg to about 200 mg of silicified microcrystalline cellulose per serving. In some cases, a supplement composition provided herein can include about 5 mg to about 40 mg of silicified microcrystalline cellulose for every gram of the supplement composition. For example, a gram of a supplement composition provided herein can contain about 5 mg to about 10 mg, about 10 mg to about 10 mg, about 10 mg to about 15 mg, about 15 mg to about 15 mg, about 15 mg to about 20 mg, about 20 mg to about 25 mg, about 25 mg to about 25 mg, about 25 mg to about 30 mg, about 30 mg to about 30 mg, or about 30 mg to about 40 mg of silicified microcrystalline cellulose.

Any appropriate method can be used to obtain a filler (e.g., silicified microcrystalline cellulose) a having an Aw of less than 0.2 for use in the supplement compositions provided herein. For example, a filler having an Aw of less than 0.2 can be obtained as described elsewhere (see, e.g., U.S. Publication No. US20140322282). In some cases, a filler having an Aw of less than 0.2 can be obtained commercially (e.g., from Mingtai Chemical Co. Ltd., Taiwan, Cat. No. M112 D).

In some embodiments, a supplement composition provided herein can include one or more glidants. Examples of glidants that can be included within a supplement composition provided herein include, without limitation, silica, stearic acid, calcium stearate, magnesium stearate, sodium stearate, glyceryl behapate (compritol), Aerosil® (colloidal silicon dioxide), and starch. In some embodiments, a supplement composition provided herein can include about 0.1 mg to about 25 mg of silica per supplement composition. For example, a supplement composition provided herein can contain about 0.1 mg to about 0.5 mg, about 0.5 mg to about 1 mg, about 1 mg to about 1.5 mg, about 1.5 mg to about 2 mg, about 2 mg to about 2.5 mg, about 2 mg to about 2.25 mg, about 2.25 mg to about 2.5 mg, about 2.5 mg to about 3 mg, about 2.25 mg to about 2.5 mg, about 2.5 mg to about 3 mg, about 3 mg to about 7 mg of silica about 7 mg to about 12 mg of silica, about 12 mg to about 15 mg of silica, or about 15 mg to about 20 mg of silica.

In some embodiments, silica can be present in an amount of about 0.02% to about 2% w/w of a supplement composition provided herein. For example, silica can be present in an amount of about 0.02% to about 0.05%, about 0.05% to about 0.1%, about 0.1% to about 0.75%, about 0.15% to about 0.2%, about 0.2% to about 0.25%, about 0.25% to about 0.3%, about 0.3% to about 0.5%, about 0.5% to about 1, about 1% to about 1.5%, or about 1.5% to about 2%. In some embodiments, silica can be present in an amount of about 0.1% to about 0.5% w/w of a supplement composition provided herein. For example, silica can be present in an amount of about 0.1% to about 0.15%, about 0.15% to about 0.2%, about 0.2% to about 0.25%, about 0.25% to about 0.3%, about 0.3% to about 0.4%, or about 0.4% to about 0.55% w/w of a supplement composition provided herein.

Any appropriate method can be used to obtain a glidant (e.g., silica) that can be included in a supplement composition provided herein. For example, silica can be obtained as described elsewhere (see, e.g., U.S. Publication No. 2006/153764). In some cases, silica can be obtained commercially (e.g., from Grace Davison Inc., Baltimore, MD; Cat. No. Syloid 244).

In some cases, a supplement composition provided herein can include magnesium stearate. When included in a supplement composition provided herein, magnesium stearate can be present in any appropriate amount. In some embodiments, magnesium stearate can be present in an amount of about 0.1% to about 10% w/w of a supplement composition provided herein. For example, magnesium stearate can be present in an amount of about 0.1% to about 2%, about 2% to about 4%, about 4% to about 6%, about 6% to about 8%, about 8% to about 10% w/w of a supplement composition provided herein. In some embodiments, magnesium stearate is present in an amount of about 0.1% to about 2% w/w of a supplement composition provided herein. For example, magnesium stearate can be present in an amount of about 0.1% to about 0.3%, about 0.3% to about 0.5%, about 0.5% to about 0.7%, about 0.7% to about 0.9%, about 0.9% to about 1.1%, about 1.1% to about 1.3%, about 1.3% to about 1.5%, about 1.5% to about 1.7%, or about 1.7% to about 2% w/w of a supplement composition provided herein.

In some cases, a supplement composition provided herein can include between about 1 mg and about 100 mg of magnesium stearate per serving. For example, a supplement composition provided herein can contain about 1 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, or about 90 mg to about 100 mg of magnesium stearate per serving. In some cases, a supplement composition provided herein can include about 5 mg to about 15 mg of magnesium stearate for every gram of the supplement composition. For example, a gram of a supplement composition provided herein can contain about 5 mg to about 8 mg, about 8 mg to about 10 mg, about 10 mg to about 12 mg, or about 12 mg to about 15 mg of magnesium stearate.

Any appropriate method can be used to obtain magnesium stearate that can be included in a supplement composition provided herein. For example, magnesium stearate can be obtained as described elsewhere (see, e.g., Chinese Patent No. CN103880645; Chinese Patent No. CN103524324, and Chinese Patent No. CN10319361). In some cases, magnesium stearate can be obtained commercially (e.g., from Peter Greven Asia, Malaysia; Cat. No. Palmstar MGST 200).

### Men's Health Supplement Formulations

The supplement compositions provided herein can be formulated for oral administration and can include any suitable excipients, flavorings, colorants, and/or other ingredients. For oral administration, tablets or capsules can be prepared with pharmaceutically acceptable excipients such as binding agents, fillers, lubricants, disintegrants, or wetting agents. In some cases, a supplement composition provided herein can be in the form of a capsule or tablet form with a fast disintegration time and/or with a delayed release. Liquid preparations for oral administration can take the form of, for example, solutions, syrups, or suspension, or they can be presented as a dry product for constitution with water, saline, or another suitable consumable liquid vehicle before use. In some cases, liquid preparations can contain pharmaceutically acceptable additives such as suspending agents, emulsifying agents, non-aqueous vehicles, preservatives, buffer salts, flavoring agents, coloring agents, and sweetening agents as appropriate. Preparations for oral administration can be suitably formulated to give controlled release of one or more compounds.

In some cases, the supplement compositions provided herein can contain a pharmaceutically acceptable carrier for administration to a mammal (e.g., a human), including, without limitation, sterile aqueous, or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Pharmaceutically acceptable carriers also can include physiologically acceptable aqueous vehicles (e.g., physiological saline) or other carriers appropriate for oral administration.

In some cases, the supplement compositions provided herein can be in the form of a capsule or tablet configured to have a unit dosage equal to the daily desired dosage for a particular mammal. For example, if a mammal desires 100 mg of a particular agent, each tablet or capsule can include about 100 mg in weight of that agent. As used herein, the term “mammals” generally refers to humans, but the term also can include domesticated mammals (e.g., dogs, cats, rats, mice, and livestock such as cows, horses, pigs, and sheep). The dosages of a particular supplement composition provided herein can depend on factors including the general health of a mammal. In some cases, a total daily dose may be prepared and administered in the form of one or more dosage forms (e.g., two tablets or capsules, three tablets or capsules, four tablets or capsules, five tablets or capsules, or six tablets or capsules). In some cases, a full serving of the supplement composition provided herein can include one or more dosage forms. For example, a full serving of the supplement compositions provided herein can be one tablet, two tablets, three tablets, four tablets, five tablets, six tablets, seven tablets, eight tablets, nine tablets, or more than nine tablets). In some cases, a full serving of the supplement compositions provided herein can be one capsule, two capsules, three capsules, four capsules, five capsules, six capsules, seven capsules, eight capsules, nine capsules, or more than nine capsules.

In some cases, the supplement compositions provided herein can contain one or more additional additives including, but not limited to, gelatin, glycerin, water, beeswax, lecithin, cocoa, caramel, titanium dioxide, or carmine.

In some cases, the supplement compositions provided herein can be provided in the form of a gel cap, soft gelatin capsule (e.g., soft gel capsule), or hard gelatin capsule. For example, a supplement composition provided herein can be encapsulated by a hard gel capsule. For oral administration, hard or soft gel capsules can be prepared by conventional means with pharmaceutically acceptable excipients such as binding agents, fillers, lubricants, disintegrants, or wetting agents. In some cases, the supplement compositions provided herein can be in the form of a softgel. Softgels are predominantly used to contain liquids containing one or more active ingredients in a dissolved or suspended state. Use of softgel formulations can enhance the bioavailability of one or more active ingredients, resulting from the fact that the ingredient is already in solution at the site of absorption, thereby permitting faster and more uniform absorption to occur. Softgels can be provided with suitable coatings that typically contain gelatin and/or suitable edible dye(s). In some cases, concentrated saccharide solutions can be used, which may optionally contain gum arabic, talc, polyvinyl pyrrolidone, polyethylene glycol (PEG) and/or titanium dioxide, lacquer solutions and suitable organic solvents or solvent mixtures, including dimethyl sulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropyl methyl-cellulose phthalate, may be used. Dye stuffs or pigments can be added to the tablets or dragee coatings or soft gelatin capsules, for example, for identification or in order to characterize combinations of active compound doses. In some cases, flavoring may be added to the softgel coat to enhance the taste. Soft gel capsule manufacturing methods are described in, e.g., U.S. Pat. No. 6,333,047.

In some cases, the supplement compositions provided herein can be in the form of a capsule or tablet that is provided in a size acceptable for a human consumer to swallow. For example, capsules can be in the range from about 250 mg to 3 g in size, or any size therebetween (e.g., about 275 mg, about 300 mg, about 350 mg, about 400 mg, about 450 mg, about 475 mg, about 490 mg, about 500 mg, about 550 mg, about 575 mg, about 600 mg, about 650 mg, about 675 mg, about 700 mg, about 750 mg, about 800 mg, about 900 mg, about 1000 mg, about 1250 mg, about 1500 mg, about 1750 mg, about 1800 mg, about 1900 mg, about 2 g, about 2.1 g, about 2.2 g, about 2.3 g, about 2.4 g, about 2.5 g, about 2.6 g, about 2.7 g, about 2.8 g, or about 2.9 g).

### Methods of Use

This document also provides methods for using the supplement compositions to treat male mammals (e.g., humans). For example, the supplement compositions provided herein can be used to increase testosterone levels and/or improve health and vitality in a male mammal (e.g., a male human). The methods can include administering to a mammal, or directing a mammal to self-administer, an effective amount of a supplement composition provided herein, where the composition includes a grape seed extract, a Eurycoma longifolia extract, a Lepidium meyenii extract, an Epimedium sagittatum extract, and a fenugreek extract, an ashwagandha extract, or a fenugreek extract and an ashwagandha extract as described herein.

In some embodiments, the supplement compositions provided herein can be used to improve health in a male mammal. For example, the supplement compositions provided herein can improve health by increasing/supporting fat-free mass, lean body mass, lean muscle tissue, healthy aging, reproductive functions, sperm counts, and/or quality of life. In some embodiments, the supplement compositions provided herein can be used to improve vitality in a male mammal (e.g., a human). For example, the supplement compositions provided herein can improve vitality in a male mammal by enhancing libido, arousal, sexual function, strength, endurance, and/or vigor. In some cases, the supplement compositions provided herein can help manage fatigue or low energy, mood changes (e.g., irritability or depression), erectile dysfunction or reduced libido, loss of muscle mass or increased body fat, thinning or loss of body hair, and/or decreased bone density or unexplained fractures.

The supplement compositions provided herein can be administered one or more times per day (e.g., one time per day, two times per day, three times per day, four times per day, or five times per day) to one or more times per week, including once every other day, once every three days, or twice a week. The skilled artisan will appreciate that certain factors may influence the dosage and timing required to effectively improve health and vitality, and increase testosterone levels in a male mammal, including but not limited to the general health and/or age of the subject, other drugs or therapies the mammal is taking, and any diseases or disorders present in the mammal. Various factors can influence the actual amount used for a particular application. For example, the frequency of administration, duration of administration, age, lifestyle, and/or combination with other drugs or therapeutic agents the mammal is taking (if any) may require an increase or decrease in the actual amount administered.

An effective duration for administering a supplement composition provided herein can be any duration that improves health, improves vitality, and/or increases testosterone levels in a male mammal without producing significant toxicity to the male mammal. In some cases, the effective duration can vary from several days to several weeks, to several months, or longer. Multiple factors can influence the actual effective duration used for a particular treatment. For example, an effective duration can vary with the effective amount, frequency of administration, age, lifestyle, and/or combination with other drugs or therapeutic agents the mammal is taking, if any.

The invention will be further described in the following examples, which do not limit the scope of the invention described in the claims.

### EXAMPLES

It would be understood that any of the suppliers and/or branded materials described herein may change; those listed in any of the Tables below are representative only.

### Example 1—Exemplary Men's Health Supplement Mixes

Tables 1A, 1B, and 1C show representative recipes for men's health supplement compositions as described herein.

TABLE 1A
First Exemplary Men's Health Supplement Mix
Ingredient

Fenugreek Extract
Ashwagandha
Eurycoma longifolia (Longjack)
Lepidium meyenii Extract (Maca)
Epimedium sagittatum Extract (Horny Goat Weed)
Grape Seed Extract

TABLE 1B
Second Exemplary Men's Health Supplement Mix
Ingredient

Ashwagandha
Eurycoma longifolia (Longjack)
Lepidium meyenii Extract (Maca)
Epimedium sagittatum Extract (Horny Goat Weed)
Grape Seed Extract

TABLE 1C
Third Exemplary Men's Health Supplement Mix
Ingredient

Fenugreek Extract
Eurycoma longifolia (Longjack)
Lepidium meyenii Extract (Maca)
Epimedium sagittatum Extract (Horny Goat Weed)
Grape Seed Extract

### Example 2—Exemplary Men's Health Supplements

Tables 2A, 2B, and 2C show representative recipes for men's health supplement compositions as described herein.

TABLE 2A
Fourth Exemplary Men's Health Supplement
Ingredient

Fenugreek Extract
Ashwagandha
Eurycoma longifolia (Longjack)
Lepidium meyenii Extract (Maca)
Epimedium sagittatum Extract (Horny Goat Weed)
Grape Seed Extract
Microcrystalline cellulose

TABLE 2B
Fifth Exemplary Men's Health Supplement
Ingredient

Ashwagandha
Eurycoma longifolia (Longjack)
Lepidium meyenii Extract (Maca)
Epimedium sagittatum Extract (Horny Goat Weed)
Grape Seed Extract
Microcrystalline cellulose

TABLE 2C
Sixth Exemplary Men's Health Supplement
Ingredient

Fenugreek Extract
Eurycoma longifolia (Longjack)
Lepidium meyenii Extract (Maca)
Epimedium sagittatum Extract (Horny Goat Weed)
Grape Seed Extract
Microcrystalline cellulose

### Example 3—Exemplary Men's Health Supplements

Table 3 shows a representative recipe for a men's health supplement composition as described herein.

TABLE 3
Seventh Exemplary Men's Health Supplement Mix

| Ingredient | (mg/serving) |
|---|---|
| Fenugreek seed extract 20% protodioscin | 50-1000 |
| Epimedium sagittatum whole plant 20% icariin HPLC | 1-20 |
| Maca 20:1 | 10-200 |
| Tongkat Ali Root 100:1 | 5-100 |
| Grape seed dry extract | 20-500 |
| Silica | 0.5-10 |
| Magnesium stearate | 1-25 |
| Microcrystalline cellulose | 1-25 |

### Example 4—Men's Health Composition Supported Men's Testosterone Levels and Improved Sexual Health

### Methods

Nine healthy men (n=12 for bloodwork), not taking any interfering medications or dietary supplements (e.g., testosterone, clomiphene, viagra, other testosterone boosting supplements, nitric oxide, or blood pressure products), participated in an 8-week, single-group, pre-post, open-label study during which they were daily administered the composition shown in Table 3. The participants had weight measurements taken, had blood drawn for marker analysis, and completed a survey that included the Energy/Fatigue subscale of the Short Form-36 (E/F SF-36) quality of life survey.

### Results

The results of this study are summarized in Table 4. Following the 8-week study period, the total testosterone level in the participants was increased by 18.0%, and the free testosterone level was increased by 18.1%. The participants also reported feeling more energetic and having a stronger libido. Both total testosterone and free testosterone showed directional increases in their levels. The percentage of total testosterone that existed as free testosterone (% Free Testosterone) was also increased (p<0.05). Sex-hormone-binding globulin (SHBG) levels were significantly decreased (p<0.05), subjective libido was significantly increased (p<0.05), and subjective energy levels, as E/F SF-36, were significantly increased (p<0.05). Moreover, a trend (p<0.10) for increased frequency of intimate activity, sleep quality, and stamina was observed.

Taken together, the results of this study demonstrated that the composition supported healthy testosterone levels with downstream effects on improving energy levels, libido, and sexual health.

Disclosed herein are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

TABLE 4
Effects on testosterone levels and sexual health

| | | | | % | |
|---|---|---|---|---|---|
| | Pre | Post | Delta | Change | d |
| Total Testosterone | 372.7 ± 166.1 | 439.9 ± 143.6 | 67.3 ± 128.4 | 18.0% | 0.434 |
| (ng/dL) | | | | | |
| Free Testosterone | 67.5 ± 18.9 | 79.7 ± 22.8 | 12.2 ± 25.8 | 18.1% | 0.586 |
| (pg/mL) | | | | | |
| SHBG (nmol/L) | 45.5 ± 17.3 | 41.0 ± 15.6* | −4.5 ± 4.4 | −9.9% | 0.272 |
| % Free Testosterone (%) | 1.83 ± 0.37 | 1.97 ± 0.37* | 0.14 ± 0.13 | 7.6% | 0.375 |
| Free Androgen Index | 33.4 ± 10.7 | 40.7 ± 10.3 | 6.2 ± 12.0 | 18.5% | 0.590 |
| (a.u.) | | | | | |
| Quality of Life (a.u.) | 5.11 ± 0.78 | 5.56 ± 0.53 | 0.44 ± 0.73 | 8.7% | 0.679 |
| Sleep Quality (a.u.) | 3.11 ± 0.78 | 3.67 ± 0.50† | 0.55 ± 0.73 | 17.9% | 0.867 |
| E/F SF-36 (a.u.) | 45.0 ± 17.5 | 61.1 ± 12.4* | 16.1 ± 15.4 | 35.8% | 1.08 |
| Stamina (a.u.) | 3.56 ± 0.88 | 3.89 ± 0.78† | 0.33 ± 0.50 | 9.4% | 0.401 |
| Libido Satisfaction (a.u.) | 4.00 ± 0.87 | 4.11 ± 1.05 | 0.11 ± 1.05 | 2.8% | 0.116 |
| Libido Strength (a.u.) | 5.00 ± 1.22 | 5.44 ± 1.51* | 0.44 ± 0.53 | 8.9% | 0.325 |
| Frequency of Intimate | 3.00 ± 1.00 | 3.56 ± 0.88† | 0.56 ±0.88 | 18.5% | 0.590 |
| Activity (a.u.) | | | | | |
| Mood Swings/ | 2.78 ± 0.67 | 2.78 ± 0.67 | 0.00 ± 1.22 | 0.0% | 0.000 |
| Irritability (a.u.) | | | | | |

*p < 0.5

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A supplement composition for improving men's health, said supplement composition comprising:
a grape seed extract,
a Eurycoma longifolia extract,
a Lepidium meyenii extract,
an Epimedium sagittatum extract, and
a fenugreek extract, an ashwagandha extract, or a fenugreek extract and an ashwagandha extract.

2. The supplement composition of claim 1, further comprising microcrystalline cellulose.

3. The supplement composition of claim 1, further comprising silica.

4. The supplement composition of claim 1, further comprising magnesium stearate.

5. The supplement composition of claim 1, wherein the fenugreek seed extract is present in the composition in an amount of from about 50 mg to about 5000 mg per serving of the supplement composition.

6. The supplement composition of claim 1, wherein the Eurycoma longifolia extract is present in the composition in an amount of from about 5 mg to about 500 mg per serving of the supplement composition.

7. The supplement composition of claim 1, wherein the Epimedium sagittatum extract is present in the composition in an amount of from about 10 mg to about 600 mg per serving of the supplement composition.

8. The supplement composition of claim 1, wherein the Lepidium meyenii extract is present in the composition in an amount of from about 10 mg to about 1000 mg per serving of the supplement composition.

9. The supplement composition of claim 1, wherein the grape seed extract is present in the composition in an amount of from about 10 mg to about 2000 mg per serving of the supplement composition.

10. The supplement composition of claim 1, wherein the ashwagandha extract is present in the composition in an amount of from about 250 mg to about 2000 mg per serving of the supplement composition.

11. The supplement composition of claim 2, wherein the microcrystalline cellulose is present in the composition in an amount of from about 1 mg to about 200 mg per serving of the supplement composition.

12. The supplement composition of claim 3, wherein the silica is present in the composition in an amount of from about 0.1 mg to about 25 mg per serving of the supplement composition.

13. The supplement composition of claim 4, wherein the magnesium stearate is present in the composition in an amount of from about 1 mg to about 100 mg per serving of the supplement composition.

14. The supplement composition of claim 1, wherein the ashwagandha extract is an ashwagandha root extract.

15. The supplement composition of claim 1, wherein the fenugreek seed extract comprises about 20% protodioscin.

16. The supplement composition of claim 1, wherein the Epimedium sagittatum extract comprises about 20% icariin as determined by HPLC.

17. The supplement composition of claim 1, wherein the supplement composition is formulated as a tablet, a capsule, or a soft gel.

18. The supplement composition of claim 17, wherein one or more tablets, one or more capsules, or one or more soft gels comprise a full serving of the composition.

19. A method of increasing testosterone levels in a male mammal, the method comprising administering to the mammal a supplement composition of claim 1.

20. A method of improving health in a male mammal, the method comprising administering to the mammal a supplement composition of claim 1.

21. A method of improving vitality in a male mammal, the method comprising administering to the mammal a supplement composition of claim 1.
